(19) United States
(12) Patent Application Publication
(10) Pub. No.: US 2014/0378939 A1
(43) Pub. Date: Dec. 25, 2014

(54) VENTED FLUID CARTRIDGE FOR MEDICAL INFUSION DEVICE

(71) Applicant: Animas Corporation, West Chester, PA (US)

(72) Inventors: William G. SAULENAS, Wayne, NJ (US); Daniel L. BAKER, Drexel Hill, PA (US)

(73) Assignee: Animas Corporation, West Chester, PA (US)

(21) Appl. No.: 14/294,273

(22) Filed: Jun. 3, 2014

Related U.S. Application Data
(60) Provisional application No. 61/838,923, filed on Jun. 25, 2013.

(51) Int. Cl. A61M 5/168

(52) U.S. Cl. CPC A61M 5/16859
USPC 604/505; 604/67

(57) ABSTRACT

The invention is generally directed toward a novel fluid cartridge for medical infusion devices. The disclosed invention describes a medicament cartridge having an internal vent structure and hydrophobic venting material to permit the equilibration of pressure between an infusion device's reservoir chamber and the external environment, without the need for a costly, difficult to maintain vent located in the housing of the infusion device.

### RELATED APPLICATIONS

This application claims priority to U.S. Ser. No. 61/838,923 filed Jun. 25, 2013, which application is incorporated herein by reference.

### FIELD OF THE INVENTION

The present invention relates, in general, to medical devices and, in particular, to fluid cartridges with protected luer connections for use with medical infusion devices.

### BACKGROUND OF THE RELATED ART

The use of drug delivery devices for various types of drug therapy is becoming more common as the automated infusion of a drug may provide more reliable and more precise treatment to a patient.

Diabetes is a major health concern, as it can significantly impede on the freedom of action and lifestyle of persons afflicted with this disease. Typically, treatment of the more severe form of the condition, Type I (insulin-dependent) diabetes, requires one or more insulin injections per day, referred to as multiple daily injections. Insulin is required to control glucose or sugar in the blood, thereby preventing hyperglycemia that, if left uncorrected, can lead to ketosis. Additionally, improper administration of insulin therapy can result in hypoglycemic episodes, which can cause coma and death. Hyperglycemia in diabetics has been correlated with several long-term effects of diabetes, such as heart disease, atherosclerosis, blindness, stroke, hypertension, and kidney failure.

The value of frequent monitoring of blood glucose as a means to avoid or at least minimize the complications of Type I diabetes is well established. Patients with Type II (non-insulin-dependent) diabetes can also benefit from blood glucose monitoring in the control of their condition by way of diet and exercise. Thus, careful monitoring of blood glucose levels and the ability to accurately and conveniently infuse insulin into the body in a timely manner is a critical component in diabetes care and treatment.

To more effectively control diabetes in a manner that reduces the limitations imposed by this disease on the lifestyle of the affected person, various devices for facilitating blood glucose (BG) monitoring have been introduced. Typically, such devices, or meters, permit the patient to quickly, and with a minimal amount of physical discomfort, obtain a sample of their blood or interstitial fluid that is then analyzed by the meter. In most cases, the meter has a display screen that shows the BG reading for the patient. The patient may then dose with the appropriate amount, or bolus, of insulin. For many diabetics, this results in having to receive multiple daily injections of insulin. In many cases, these injections are self-administered.

Insulin pumps are generally devices that are worn on the patient's body, either above or below their clothing. Because the pumps are worn on the patient's body, a small and unobtrusive device is desirable. Some devices are waterproof, to allow the patient to be less inhibited in their daily activities by having to remove their drug infusion device while showering, bathing, or engaging in various activities that might subject their infusion device to moister, such as swimming In such devices, it would be desirable to have a structure and method for verifying proper function of venting system within the device, since vents are typically passive devices that have no means for self-diagnostic checks to verify function has been compromised (i.e. intentional or unintentional obstruction of vent opening(s)). Further, it would be desirable to be able to alert the user of abnormal pressure differentials within their device that may cause erratic or unintentional drug delivery. Finally, it would be desirable for a drug infusion device to incorporate means for detecting the altitude at which the device is located, to avoid problems associated with air travel and sporting activities such as mountain climbing, skydiving, etc. that patients may wish to engage in without having to forego the use of their drug infusion device for concerns over erratic or unintentional drug delivery due to rapid pressure changes in and around the device.

It is therefore desirable to provide method and system for delivering insulin that equilibrates internal compartments of the infusion device to changing atmospheric pressure in the most reliable and efficient manner possible.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1A shows an embodiment of a fluid cartridge typically used in a medical infusion device, according to the prior art, in side plan view.

FIG. 1B shows an embodiment of a fluid cartridge typically used in a medical infusion device, according to the prior art, in cross-sectional view.

FIG. 2A shows an embodiment of a fluid cartridge according to the present invention, in side plan view.

FIG. 2B shows an embodiment of a fluid cartridge according to the present invention, in cross-sectional view.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient,” “host,” “user” and “subject” refer to any human or animal subject and are not intended to limit the devices or methods to human use, although use of the subject invention in a human patient represents a preferred embodiment.

The present invention relates to cartridges that are used in drug delivery devices, including but not limited to insulin pumps. For purposes of illustration, this specification will refer to the structure and use of cartridges that store a quantity of medication and are inserted into a drug delivery device, such as an insulin pump, so that the medication can be infused into a patient.

Insulin pumps are devices which are typically worn on the patient's body, either above or below their clothing. These relatively small, unobtrusive devices typically store a quantity of insulin in a replaceable cartridge and include a processing unit, a display screen, and input functions such as buttons or a keypad. Such pumps may include the ability to run multiple insulin delivery programs, such as basal and bolus programs, to eliminate the need for injections of insulin via needles and syringes, by providing medication via an infusion device that can be worn by the patient for an extended period of time, usually in the range of 1-3 days.

Patients using insulin pumps typically have the ability to program insulin delivery times and amounts into their pump's software, and enter their blood glucose (BG) values into the pump via a data input system to deliver boluses of insulin in response to their activities, such as exercise and meal intake. Alternatively, the BG meter and pump may be in communication to permit the meter to transmit the BG reading to the pump along with a recommended bolus value, or to permit the pump or user to determine the appropriate bolus of insulin, if any.

Most portable insulin infusion pumps do not have a means for detecting air within the drug reservoir or line set. Such drug delivery systems operate under the premise that there is no air in the drug reservoir. Dosing controllers assumes that there a linear displacement of the drive mechanism that advances the cartridge plunger, thereby displacing a known volume of drug based on the constant area geometry of the cartridge barrel.

Typically, product labeling for these pump systems emphasizes the need to eliminate all air from the drug reservoir and line set prior to commencement of drug delivery. However, if air is present within the drug reservoir it will inherently lead to under infusion at some point during therapy. In addition, even when all precautions are taken to remove air from the drug reservoir, environmental factors such as changes in temperature and/or ambient pressure can cause air to come out of solution, which results in the formation of air bubbles in the drug reservoir or line set.

A further complication for portable infusion pump designers is that some portable infusion pumps are intended to be waterproof, to allow the patient wearing the device to maintain an active lifestyle and to allow the pump to be used during normal, daily activity, such as bathing. This is an attractive feature for people with lifestyles that benefit from continuous drug infusion (i.e. infusion of insulin for people with diabetes). Such devices must be designed with sealed enclosures/housings to prevent ingress of water. To avoid the development of pressure differentials between the external environment and the sealed compartment that houses the drug reservoir, most waterproof pumps incorporate hydrophobic vents that allow passage of air, but not fluids (within certain limitations of pressure differential).

Most portable drug infusion pump reservoirs developed from the most basic method of delivering medication—a standard syringe. Therefore, the reservoir is typically comprised of two major components; a cylindrical barrel, with a connector integrated into the distal end for attachment of an infusion line set, and a movable plunger with an elastomer seal. The plunger is inserted into the open proximal end of the barrel to form a closed volume. To deliver drug, a mechanically driven piston is advanced forward, which in turn advances the cartridge plunger forward, reducing the internal volume of the cartridge, thus displacing fluid. Typically, the piston (part of the durable device) is not mechanically interlocked with the cartridge plunger because there is no need to retract the plunger once the cartridge has been filled and subsequently installed in the pump.

If the pump piston is not interlocked with the cartridge plunger, there is a risk of unintentional delivery of drug if a positive pressure differential were to develop between the chamber that houses the reservoir and the external environment (location of infusion site). A positive pressure differential would impart a resultant force on the plunger which is directly proportional to the cross-sectional area of the drug reservoir's internal volume. If the resultant force exceeds the sustaining force of the cartridge plunger it will advance the plunger forward and thus deliver drug. Thus, it is a consideration in the design and manufacture of a drug infusion pump to ensure that the chamber in which the drug cartridge resides remains at or close to the external, ambient air pressure. To date, this has been done via costly, replaceable vents built directly into the reservoir chamber. These vents can clog over time, due to contaminants in air and water (dust, dirt, etc.) requiring that they be changed. Changing these vents often require that the infusion device be returned to the manufacturer, thereby depriving the patient of the device's use for a period of time, simply to have the vent changed.

It has been found that a novel cartridge design that provides an integrated venting mechanism to permit equalization of the chamber in which it resides with the external, ambient environment can eliminate the need for a costly, difficult to change vent in the infusion device's housing. Further, but incorporating the vent into the fluid cartridge, it is changed whenever the cartridge is changed, obviating the need for the device to be returned to the manufacturer for service. Moreover, by incorporating a fresh, clean vent into the system with each cartridge change, there is increased reliability and assurance that the chamber in which the cartridge resides will be adequately vented to avoid unintended fluid delivery.

Most diabetics that use an insulin infusion device purchase their insulin separately from the cartridges that they insert into their infusion pump. In order to fill the cartridge, they insert a needle attached to the cartridge into an insulin vial and pull back on an extractor. Once filled, they insert the cartridge into the insulin pump and attach an infusion set to the cartridge. The portion of the infusion set that attaches to the cartridge is generally referred to as the luer. The luer connects to tubing, referred to as the lineset tubing that terminates in a cannula that is inserted under the skin of the patient to permit the infusion of the insulin. The cannula is generally held in place with an adhesive patch, to avoid accidental dislodgement.

The cartridge, plunger and the extractor are typically formed from implantable grade plastic, such as long-term implantable plastics including, but not limited to polyethylenes, polyetheretherketones (PEEK) and bioabsorbables-polylactic acid (PLA), polyglycolic acid (PGA) and their copolymers. In instances where the materials do not need to be of an implantable grade, those skilled in the art will readily recognize that numerous additional plastics that are suitable for use, including various polyethylene and polyester acrylates and resins. Extrusion, injection molding, and casting are typical manufacturing methods employed for this process.

In general, portable external infusion devices, such as portable insulin pumps, are well-known. Users, such as diabetics, wear these devices in their clothing, e.g., on a belt or in a clothing pocket. In order to allow the user to enjoy a full range of activities, including for example, swimming, and outdoor activities, it is necessary for the device to resist ingress of water, which could damage the device's internal electronic components.

The need for such water hermeticity is complicated by an additional need to ensure pressure equilibrium between the interior of the device and atmosphere, in order to avoid pressure gradients inside the device that could adversely impact the delivery of liquid medication, such as insulin. A need for rapid pressure equalization can arise, for example, when the user flies in an airplane, and pressure in the airplane cabin fluctuates due to ascent or descent of the airplane. Such a fluctuation in cabin pressure could cause pressure inside an insulin pump casing to rapidly exceed cabin pressure, which could result in a sudden unexpected and undesirable infusion of insulin to the user.

Conventional infusion pumps typically include a casing defining a single housing. The housing encloses, within a single external wall, a medicinal reservoir, a driving mechanism, electronic circuitry for controlling the driving mechanism, a battery, o-rings sealing a battery door and a reservoir door, and vents, to allow passage of air, but prevent passage of liquid. These vents allow pressure within the casing to equalize with atmospheric pressure.

Notwithstanding these features, the conventional single housing device has at least one major drawback, namely that ingress of water, spillage of insulin, or any other ingress of liquid, due to a mechanical failure, or an operator error, e.g., forgetting to securely shut the reservoir door or battery door after changing the reservoir or the battery, allows liquid to reach electric components and the sensitive electronic circuitry, which can damage the components and circuitry permanently, or at least cause the device to malfunction.

Moreover, while some known infusion pumps include a casing with separate compartments, these compartments are not hermetically sealed from one another, so water leaking into one compartment also can flow into the other compartment(s), with the same risk to electronic components and circuitry.

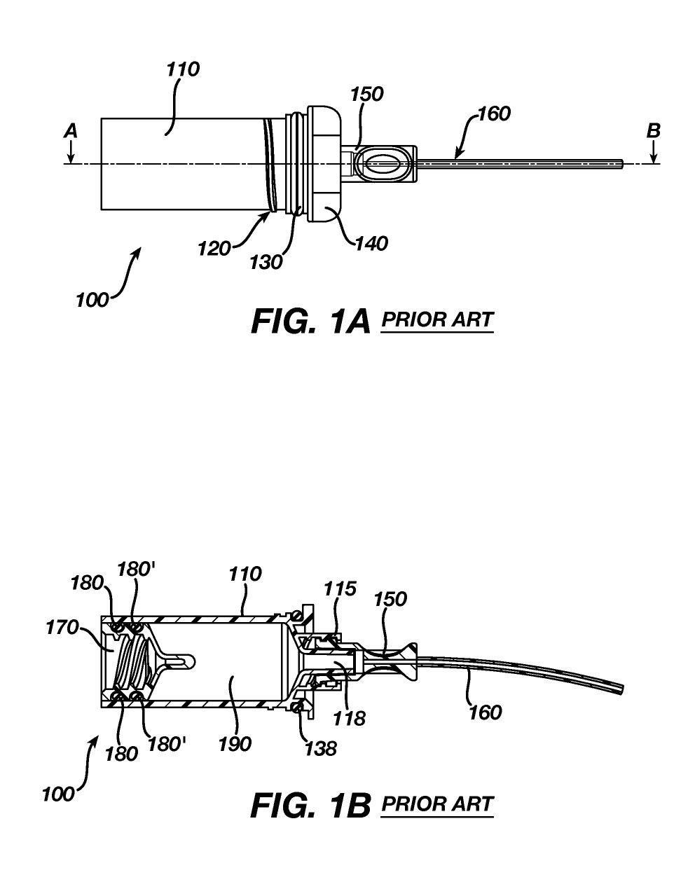

FIGS. 1A and 1B refers to a prior art device that is exemplary of cartridges presently used to store medication and use with an infusion device. The cartridge 100 has a body 110 with a reservoir 190 for storing fluid. The cartridge body 110 has an external thread 120 to allow the cartridge to be screwably secured within a cavity within the drug delivery device (not shown).

A plunger 170 is inserted into the reservoir 190 to expel the fluid out of the cartridge via a nipple 118. The head of the plunger 170 is generally equipped with one or more O-rings 180, 180′ to minimize leakage from between the plunger 170 and the interior of the reservoir 190. An infusion set (partially shown) comprising a luer 150 and lineset tubing 160 is secured to the cartridge to permit fluid communication by screwing the luer 160 into a luer connector 115 that has internal threads for receiving the luer 160. Once attached, the luer is secured to the cartridge using a cartridge cap 140. An O-ring 130 is typically included to maintain a seal between the cartridge 100 and the cavity in which it is disposed within a drug infusion device.

The prior art device, when inserted into the reservoir chamber of an insulin pump, can become “vacuum locked” within the device due to the creation of a seal between the o-rings and the walls of the reservoir chamber. One solution presently employed in the art is to include a hydrophobic vent in the reservoir chamber that is capable of equilibrating the pressure in the compartment while retaining the integrity of the device against water or liquid incursion.

The prior art solution, however, requires that the hydrophobic vent be replaced regularly as it can become clogged with normal environmental contaminants (dust, dirt, etc.) during normal use. To change the vent, the infusion device must be sent to the manufacturer to replace the vent. Hydrophobic vents designed for minimal clogging over an extended period of time are expensive. The cost of the vent and the time and resources required by the manufacturer to replace the vent coupled with the inconvenience to the patient of being without their infusion device for a period of time suggests a strong need for a more cost-effective and efficient solution.

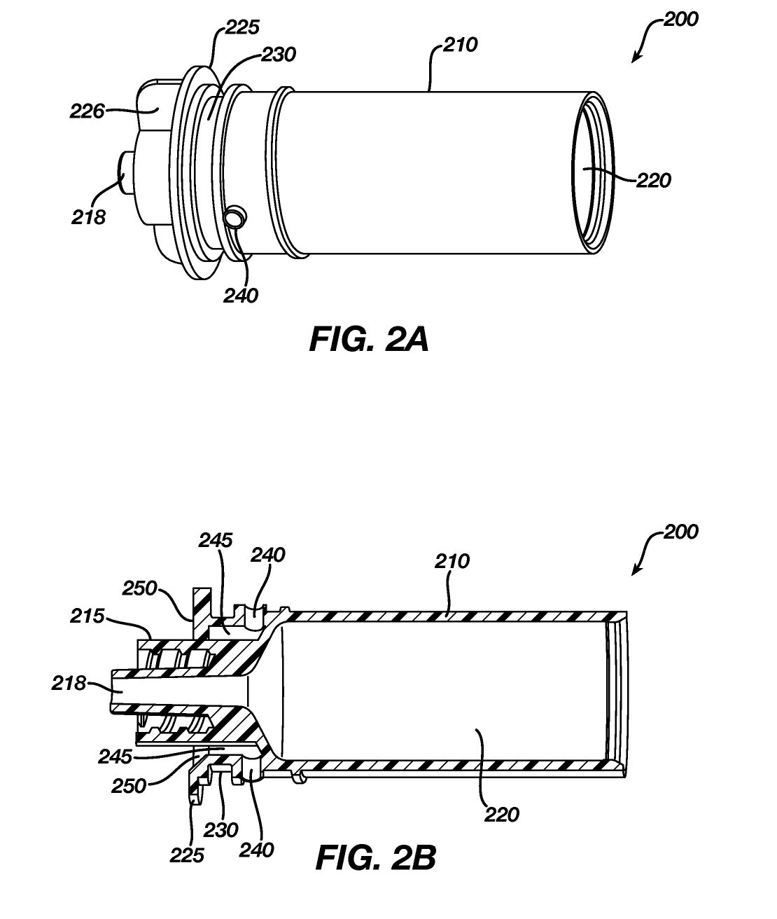

A device according to FIGS. 2A and 2B overcomes the shortcomings of the prior art solution by providing a vented drug cartridge for use in a drug infusion pump. Drug infusion pumps contemplated for use with the present invention are exemplified by the OneTouch® Ping® insulin infusion pump, sold by Animas Corporation of West Chester, Pa.

FIGS. 2A and 2B show an embodiment of the invention in which a cartridge 200 for containing a quantity of medication has an outer wall 210 that has a diameter that is just slightly smaller than a reservoir chamber of an insulin infusion device in which the cartridge is intended to be inserted. The cartridge 200 has a cavity 220 therein for storing medication and for receiving a plunger (now shown) that is advanced into the cavity 220 by a drive mechanism (not shown) to expel fluid through the outlet port 218 that extends through a luer connector 215 at a first end of the cartridge 200. The luer connector 215 may be internally or externally threaded to receive an end cap 226 to hold the luer of an infusion set in-place (not shown).

The first end of the cartridge 200 also may include a sealing ring 225 proximate or adjacent to a groove 230 where an o-ring (not shown) may be placed. The sealing ring 225 and the o-ring in the groove 230 create a substantially water and air tight seal between the environment external to the infusion device and the reservoir chamber of the device in which the cartridge 200 is inserted for use.

The side wall 210 of the cartridge 200 also includes an internal vent port 240 that is in fluid communication with an external vent port 250 via a bore or channel containing a hydrophobic vent material 245. The external vent port 250, hydrophobic vent material 245, and internal vent port 245 combine to create a channel through which the atmospheric pressure in the reservoir chamber of the infusion device can equilibrate with the external environment without the incursion of water or other liquids into the reservoir chamber. Further, by incorporating the hydrophobic vent material 245 into the cartridge, a new, clean vent is installed each time the patient or healthcare provider changes cartridges—for the typical Type 1 diabetic using insulin pump therapy, for example, this may be every 3-5 days. This greatly reduces the cost associated with the hydrophobic vent material, since the material need only remain clean and unobstructed for a few days to a week, or so, versus the many months to years between vent servicing that is typical of current-generation insulin infusion devices.

Preferably, the hydrophobic vent material 245 is selected so that a water entry pressure (WEP) of the hydrophobic vent material 245 significantly exceeds a fluid pressure at a selected depth, i.e., the depth to which they can reasonably expect to be exposed upon immersion in water. For example, in the case where a test pressure of 5.2 psi is requested (i.e., water pressure at a depth of 12 feet below the surface), a selected WEP of approximately 10 to 15 psi provides a preferable design margin.

It is likewise preferable that once a suitable WEP is selected, the hydrophobic membrane is selected from among those providing the highest available air flow rate, in order to achieve, along with the desired water resistance, the ability to equalize pressure across the membrane as rapidly as possible, preferably within seconds.

Various suitable materials exist from which the hydrophobic vent material 245 can be fabricated. Useful materials include those that are porous plastics and that are compatible with sterilization processes such as ETO, steam sterilization and the like. Suitable materials include polytetrafluoroethylene (“PTFE”), polyethylene, polyvinylidene fluoride (“PVDF”), ultra-high molecular weight polyethylene (“UPE”), and the like and combinations thereof For example, PTFE is a widely used material in medical venting and gas filtration. It is an inert material that offers excellent flow properties and high chemical resistance. Dimensional instability of cut shapes of this membrane type can cause difficulties in robotic handling in over-molding operations. PTFE is incompatible with gamma or E-beam sterilization because chain scission causes loss of integrity when the material is exposed to ionizing radiation.

PVDF is a durable material that offers good flow properties and broad chemical resistance. It is available in both natural and super-hydrophobic forms.

UPE is a more recent entry into the medical venting and gas filtration market. It is a naturally hydrophobic material that offers excellent flow properties and broad chemical resistance.

The vent may be a cylindrical plug that is inserted into a hole in the skirt wall of the cartridge barrel and retained by adhesive, heat stake, co-molding or the like or combinations thereof The plug may be made from sintering plastic powder spheres of controlled and consistent diameter under conditions of heat and pressure. The sintering process creates a structure having a uniform, rigid external geometry with a known, consistent void path throughout. Sintering will cause the contact points of the spherical particles to fuse and solidify leaving open, but torturous, pathways of airspace between the spheres. Air with a low dynamic viscosity (1×10⁻⁵ Pa*s) can flow freely and vent through the resulting porous structure. Water with a higher viscosity (1×10⁻³ Pa*s) will require more energy or pressure to pass through the structure.

Hydrophobic vented closures balance the air pressure between the pump interior and the atmosphere and prevent the ingress of water or contamination. By controlling the spherical size of the original plastic powder, the hydrophobicity of the plastic resin employed, and the degree of sintering, a porous structure can be created to maximize air transmission while restricting water ingress through the vent to suit the use to of the cartridge syringe in an insulin pump device.

As yet another alternative, modified acrylic membrane treated to be hydrophobic is an economical choice for venting applications. It is oleophobic, hydrophobic, and chemically compatible. Another alternative is provided by use of GORE™ self-adhesive vent tape to be applied over the hole in the cartridge barrel skirt wall to create a controlled porous structure.

Thus, by providing a cartridge that has an integrated, internal vent, drug infusion pumps that do not require a reservoir chamber vent can be manufactured while still retaining the ability to equilibrate pressure while retaining resistance to water incursion.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. A medicament cartridge, comprising:
a housing having an open proximal end, a distal end, and a cavity therein defining a reservoir;
a plunger configured to slidably insert into the proximal end of the cartridge;
a luer connector at the distal end of the cartridge;
a fluid outlet port in fluid communication with the reservoir at the distal end of the cartridge;
at least one groove about the circumference of the housing configured to receive an pliant O-ring;
a sealing ring between the at least one groove and the distal end of the housing; and
a vent port comprising an external port extending through the sealing ring and an internal port extending through the housing.

2. The medicament cartridge of claim 1, comprising a vent material disposed in the vent port.

3. The medicament cartridge of claim 2 wherein the vent material is hydrophobic.

4. The medicament cartridge of claim 3 wherein the vent material comprises polyvinylidene fluoride, polytetrafluoroethylene, ultra-high molecular weight polyethylene, a hydrophobically treated modified acrylic, or mixtures thereof

5. The medicament cartridge of claim 4, wherein the vent port has a WEP of from about 10 psi to about 15 psi.

6. A method for resisting fluid incursion in a medical infusion device, comprising:
providing a reservoir chamber in the medical infusion device configured to receive a medicament cartridge;
providing the medicament cartridge, the medicament cartridge comprising a housing having an open proximal end, a distal end, a cavity therein defining a reservoir, a luer connector at the distal end of the cartridge, a fluid outlet port in fluid communication with the reservoir at the distal end of the cartridge, at least one groove about the circumference of the housing configured to receive an pliant O-ring, a sealing ring between the at least one groove and the distal end of the housing, and a plunger configured to slidably insert into the proximal end of the cartridge, and a vent port comprising an external port extending through the sealing ring and an internal port extending through the housing, wherein the sealing ring has a diameter less than or equal to the diameter of the reservoir chamber;
inserting the medicament cartridge in the reservoir chamber;
equilibrating the atmosphere in the reservoir chamber with the atmosphere external to the medical infusion device.

7. The method of claim 6, comprising inserting a vent material into the vent port, prior to inserting the cartridge into the medical infusion device.

8. The method of claim 7 wherein the vent material is hydrophobic.

9. The method of claim 8 wherein the vent material comprises polyvinylidene fluoride, polytetrafluoroethylene, ultra-high molecular weight polyethylene, a hydrophobically treated modified acrylic, or mixtures thereof.

10. The method of claim 9 wherein the vent port has a WEP of from about 10 psi to about 15 psi.
